(19) United States
(12) Patent Application Publication
(10) Pub. No.: US 2013/0177661 A1
(43) Pub. Date: Jul. 11, 2013

(54) Saussurea Involucrate Extract, Pharmaceutical Composition and Use for Anti-Fatigue and Anti-Aging

(71) Applicants: Yi-Lin Chen, Yilan County (TW); Hsin-Sheng Tsay, Yilan County (TW); Tzong-Der Way, Yilan County (TW); Po-Lei Lee, Yilan County (TW); Chao-Yuan Yu, Yilan County (TW); Chien-Hsu Chen, Yilan County (TW)

(72) Inventors: Yi-Lin Chen, Yilan County (TW); Hsin-Sheng Tsay, Yilan County (TW); Tzong-Der Way, Yilan County (TW); Po-Lei Lee, Yilan County (TW); Chao-Yuan Yu, Yilan County (TW); Chien-Hsu Chen, Yilan County (TW)

(73) Assignee: NATIONAL ILAN UNIVERSITY, Yilan County (TW)

(21) Appl. No.: 13/344,019

(22) Filed: Jan. 5, 2012

(51) Int. Cl. A61K 36/28; A61P 29/00

(52) U.S. Cl. 424/725

(57) ABSTRACT

Disclosed are a Saussurea involucrate extract, pharmaceutical composition and use thereof for anti-fatigue and anti-aging. In the embodiments of the present invention, a low dose of ethyl acetate fraction of Saussurea involucrate (preferably 30 mg/kg) and Rutin (preferably 30 mg/kg) can inhibit MDA expression level and increase GPx activity. Additionally, both decrease the expression of COX-2, PARP and caspase-3, via downregulation of NF-kappaB, resulting in neuroprotection. Further, syringe feeding of Saussurea involucrate extract is also performed in aging mouse model. In the future, Saussurea involucrate extract of the present invention can be used in neuroprotection, particularly in treatment or prevention of cerebral diseases, nerve diseases, neurodegenerative diseases, chronic neurodegenerative diseases, aging, and fatigue, caused by oxidative injury.

### BACKGROUND OF THE INVENTION

1. Field of the Invention

The present invention relates to a Saussurea involucrate extract and use thereof, particularly to a Saussurea involucrate extract, pharmaceutical composition, and use for anti-fatigue and anti-aging.

2. The Prior Arts

In recent years, neurodegenerative diseases have become major diseases for influence on human health and life quality and caused a high death rate. Traditional neurodegenerative diseases include Parkinson's disease, amyotrophic lateral sclerosis (ALS), multiple sclerosis, Huntington's disease, Alzheimer's disease (AD), diabetic retinopathy, multi-infarct dementia, and macular degeneration etc. Due to increasing human lifespan, prevalence of neurodegenerative diseases has been deeply aware. Highly relative incidence of these diseases (between 2-15%, 70-year-old group) has become a serious medical, social and financial burden for patients, healthcare employees and the pubic. The rate of a person, who is diagnosed as suffering from neurodegenerative diseases, dramatically increases in association with its age, particularly the patients suffering from Alzheimer's disease. Statistics indicates that there are 24,000,000 patients suffering Alzheimer's disease in the world, and the patient numbers still increase. Alzheimer's disease (AD) is an age-related neurodegenerative disorder of central nervous system and may result in memory loss and dementia. Some of the associated pathogenic changes are amyloid peptide, lipofuscin and malomdialdehyde (MDA) aggregation, excitotoxicity, oxidative stress and inflammation. In neuropathology, senile plaques and neurofibrillary tangles are found in patient's brain. Free radicals and peroxides are found to be one of the major causes for senile plaques in Alzheimer's disease. Initially, lipofuscins in mitochondria are an oxidative polymerization product of proteins and lipids, and they gradually accumulate in axon to cause neurofibrillary tangles and finally influence the normal function of the brain. As indicated in many studies, peroxides promote the rate of lipofuscin accumulation, and antioxidants can attenuate lipofuscin accumulation. Therefore, if a drug is able to efficiently eliminate senile plaques and neurofibrillary tangles, it will also be considered as a treatment for Alzhemer's disease. Though the palliative treatment has been developed, there is no method can completely cure Alzhemer's disease and recover its normal function.

Generation of free radicals highly relates with aging. Among the related theories, free radical theory is the most well-known one in this field. A free radical is any atom or molecule that has a single unpaired electron in an outer shell. This molecular structure is extremely unstable and highly reactive. Among all free radical species, the oxygen free radical is the most important one for human health, which is generated in two different ways. The first way is by production through regular metabolism within the body. In invasion of bacteria, fungi, viruses, or other foreign objects, immune system would encompass foreign objects and thus generate oxygen free radicals. Alternatively, other irregular factors such as radiation, UV, smoking, air pollution and psychological pressure may result in generation of free radicals. Upon the amount of free radicals within body exceeds that the immune system can defense, oxidative stress will be produced, which may cause various diseases and aging. Therefore, generation of free radicals results in a chain reaction that can induce oxidation of proteins, carbohydrates, lipids, and nucleic acids, and form peroxidative lipid accumulation to damage cell membranes, proteins and nucleic acids within body, resulting in losing normal function and cause diseases. If ability of defense system within body can be promoted to degrade reactive oxygen species (ROS), cellular damage thus can be reduced, and the lifespan also can be extended.

The antioxidant defense system in organism can be classified into primary defense system and secondary defense system. Primary defense system includes, for example, vitamin C, vitamin E, glutathione, and antioxidant enzymes such as superoxide dismutase (SOD), catalase, and glutathione peroxidase (GPx) that can directly eliminate ROS or inhibit oxidation of ROS. Secondary defense system includes lipolytic enzymes, proteolytic enzymes, and DNA repair enzymes, which are respectively responsible for oxidative products of ROS, repair of injured cells and removal of free radical derivatives that primary defense system can not degrade. In addition to vitamin C and vitamin E, diverse enzymes have been approved to possess antioxidant activity. For example, Coenzyme Q10 (CoQ10) is a lipid soluble vitamin within the cell, which is considered to relate to various neuron degenerated diseases, such as Parkinson's disease and Alzheimer's disease. Functionally, CoQ10 is a critical antioxidant and able to regenerate vitamin E within the lipoprotein. CoQ10 also can promote metabolism of cell respiration, and play an important role for free radical elimination. Alpha-lipoic acid is an internal antioxidant and able to directly eliminate ROS. It is found that alpha-lipoic acid is helpful in some human diseases such as cataracts, diabetes, and acquired immunodeficiency syndrome.

In the present generation, high pressure and irregular diet tend to influence health. Considering importance of health, functional food for anti-aging and anti-fatigue has become a trend. In recent years, it has revealed that aging and fatigue highly relate to free radicals and antioxidants. Once antioxidant system in body is unable to eliminate excessive free radicals, oxidative injury will accumulate and unbalance mechanism of antioxidant, finally influence the physiological function and cause aging, genetic mutation and even death.

It is known that Saussurea involucrate contains very high levels of flavonoid compounds. Flavonoid is a widely discussed antioxidant compound existed in vegetables, beans and crops, and is approved to have effects on stroke prevention and neuroprotection. In addition, flavonoids can reduce some cytokines (e.g. interleukin-6 and TNF-α) released by neuroglial cell while suffering a brain disease, so as to attenuate inflammation and to promote repairing central nervous system disorder, particularly for treatment in alzheimer's disease (McCarty, 2006).

### SUMMARY OF THE INVENTION

It is an unsolved object that extraction of active compounds from Saussurea involucrate for anti-fatigue or use in treatment, prevention of brain or nervous diseases caused by oxidative injury, and further to realize the related biochemical mechanism.

An objective of the present invention is to provide a Saussurea involucrate extract for anti-fatigue and anti-aging, produced by following steps: (A) extracting Saussurea involucrate with a first solvent and evaporating the first solvent to form a solid phase; (B) adding a predetermined amount of water into the solid phase to form a suspension; (C) adding a second solvent into the suspension to form an aqueous layer and an organic layer; and (D) evaporating the organic layer to obtain the Saussurea involucrate extract. Preferably, the first solvent in step (A) is methanol, the second solvent in step (B) is pentane, ethyl acetate or butanol, the Saussurea involucrate extract at least comprises a flavonoid compound which is preferably Rutin.

Another objective of the present invention is to provide a use of Saussurea involucrate extract in the manufacture of a medicament for anti-fatigue and anti-aging, comprising the steps of administering a therapeutically effective amount of the Saussurea involucrate extract to a subject for anti-fatigue and anti-aging in need thereof, wherein the Saussurea involucrate extract can inhibit malomdialdehyde (MDA) expression, promote the removal of lactic acid, increase the superoxide dismutase (SOD) activity, increase the glutathione peroxidase (GPx) activity, and decrease cyclooxygenase 2 and caspase-3 expression of the subject.

Yet another objective of the present invention is to provide a pharmaceutical composition for anti-fatigue and anti-aging, comprising the Saussurea involucrate extract and a pharmaceutically acceptable diluent, excipient or carrier. The pharmaceutical composition is in the form selected from the group consisting of an intravenous injection agent, nasal agent, inhalation agent, sublingual tablet, suppository, topical injection agent, transdermal medication, oral medicine, ophthalmic agent, muscular injection agent, subcutaneous injection agent, intracutaneous injection, slow release formulation and control release formulation.

In the embodiments of the present invention, by subcutaneous administration of ethyl acetate fraction of Saussurea involucrate extract, aging mouse model is established and exhibits that low concentration of ethyl acetate fraction of Saussurea involucrate extract can effectively prevent D-gal induced aging. In addition, it is demonstrated in weight-loaded swimming test that ethyl acetate fraction of Saussurea involucrate extract can increase lactic acid elimination to combat fatigue. In the future, Saussurea involucrate extract of the present invention can be used in neuroprotection, particularly in treatment or prevention of cerebral diseases, nerve diseases, neurodegenerative diseases, chronic neurodegenerative diseases, aging, and fatigue, caused by oxidative injury. In weight-loaded swimming test, ethyl acetate fraction of Saussurea involucrate extract can increase lactic acid elimination to combat fatigue.

### BRIEF DESCRIPTION OF THE DRAWINGS

The present invention will be apparent to those skilled in the art by reading the following detailed description of preferred embodiments thereof, with reference to the attached drawings, in which:

FIGS. 1(A)˜(C) show that the effect of Saussurea involucrate extract (30 mg/kg for 42 consecutive days) on neuronal damage analysis in D-galactose-treated (aged) mice, and serum superoxide dismutase (SOD), glutathione peroxidase (GPx) and malomdialdehyde (MDA) expression levels. Four groups represent PBS, D-galactose, Saussurea involucrate extract+D-galactose and Saussurea involucrate extract treated, respectively (*p<0.05 versus the D-galactose group).

FIG. 2 shows the effect of Saussurea involucrate extract (30 mg/kg) on the D-gal-treated mice, and COX-2 expression level by western blotting.

FIGS. 3(A)˜(D) show the effect of Saussurea involucrate (30 mg/kg) for apoptosis in the hippocampus of D-gal-treated mice, and cleaved caspase-3 expression level by Immunohistochemistry (IHC) staining. FIGS. 3(A)˜(D) represent PBS, D-galactose, Saussurea involucrate+D-galactose and Saussurea involucrate-treated, respectively.

FIGS. 4(A)˜(C) show that the effect of Rutin (30 mg/kg for 42 consecutive days) on neuronal damage analysis in D-galactose-treated (aged) mice, and serum superoxide dismutase (SOD), glutathione peroxidase (GPx) and malomdialdehyde (MDA) expression levels. Four groups were treated with PBS, D-galactose, Saussurea involucrate extract+D-galactose and Saussurea involucrate extract, respectively (*p<0.05 versus the D-galactose group).

FIG. 5 shows the effect of Rutin (30 mg/kg) on the D-gal-treated mice, and NF-kappaB and COX-2 expression levels by western blotting.

FIGS. 6(A)˜(D) show the effect of Rutin (30 mg/kg) for apoptosis in the hippocampus of D-gal-treated mice, and cleaved caspase-3 expression level by Immunohistochemistry (IHC) staining. FIGS. 6(A)˜(D) represent PBS, D-galactose, Rutin+D-galactose and Rutin-treated, respectively.

### DETAILED DESCRIPTION OF THE PREFERRED EMBODIMENTS

### Definition

The term “neuroprotection” as used herein, means protecting neurons within brain, central nervous system or peripheral nervous system (preferably within brain or spinal cord) so as to avoid cell death or damage. Preferably, the neurons are protected to prevent from death or damage caused by oxidative stress (e.g. oxygen free radicals). Further, the term “method of neuroprotection” as used herein, means a method for protecting neurons after brain, central nervous system or peripheral nervous system injury (e.g. protecting neurons to avoid cell death or damage), and the damage is caused by chemical, toxic, epidemic, radiative, or traumatic Injury. Preferably, the method of neuroprotection means a method for protecting neurons after brain, central nervous system or peripheral nervous system injury, and not limited to any cause.

The term “neurodegenerative disease” as used herein, includes Alzheimer's disease (AD), Niemaoh-Pickdisease (NPD), diffuse Lewy body disease, progressive supranuclear palsy, multisystem degeneration, Shy-Drager syndrome; neurodegeneration related diseases including chronic epilepsy; motor neuron diseases including amyotrophic lateral sclerosis (ALS), degenerative ataxias, cortical basal degeneration, ALS-Parkinson's-Dementia complex of Guam, subacute sclerosing panencephalitis, Huntington's disease, Parkinson's Disease, synucleinopathies (including multiple system atrophy), primary progressive aphasia, striatonigral degeneration, Machado-Joseph disease/spinocerebellar taxia type 3, olivopontocerebellar degeneration, Gilles De La Tourette's disease, bulbar and pseudobulbar palsy, Kennedy Disease, Multiple Sclerosis, primary lateral sclerosis, familial spastic paraplegia, Werdnig-Hoffman disease, Kugelberg-Welander disease, Tay-Sachs disease, Sandhoff&apos; s disease, familial spastic disease, Wohlfart-Kugelberg-Welander disease, spastic paraparesis, progressive multifocal leukoencephalopathy, and familial dysautonomia. Preferably, “chronic neurodegenerative disease” as used herein, includes Alzheimer's disease, Parkinson's Disease, Multiple Sclerosis, or Cerebral Palsy.

### Materials and Methods

The wild plant of S. involucrata used in the present invention was a gift from Biopure Biotechnology (Changhua, Taiwan). Twenty grams of dried and powdered aerial parts, including flower, of S. involucrate was extracted with 100 mL of methanol three times under reflux for 2 h, respectively. The methanol extracts (SI-1) were combined, and the solvent was evaporated in vacuo to give a deep brown syrup. The syrup was resuspended in water and then partitioned successively with pentane, ethyl acetate (SI-2) and n-butanol (SI-3) to leave a water layer (SI-4). The solvents were evaporated respectively, and the residues were used throughout the present invention.

Reverse-Phase High-Performance Liquid Chromatography (HPLC) Analysis of Flavonoids in S. involucrata.

The determination of flavonoids from S. involucrata was carried out by HPLC with a photodiary detector. The HPLC system consisted of a Shimadzu LC-20AT solvent delivery system, equipped with a SPD-M20A photodiode array detector, set at 270 nm. Samples were injected with SiL-20A autosample to separate on the TSK-Gel ODS-100S column. The column was maintained at an ambient temperature of 25° C. The flow rate of the system was 1.0 mL/min. The mobile phase consisted of solvent A (0.3% formic acid) and solvent B (acetonitrile). The elution profile for A was 0-10 min, with a linear gradient change of 0-5%; 10-40 min, with a linear gradient change to 55%; and maintained for another 10 min with a post run time to equilibrate the column and for the baseline to return to the normal and initial working conditions.

### Embodiment 1

### D-Galactose Aging Animal Treatment

There is a “Lipofuscin accumulation theory” among various aging theories. The lipofuscin contains 30˜70% of protein, 20˜50% of lipids, 4˜7% of carbohydrates and trace elements, which are mainly accumulated in myocardial cells, retinal cells, neuron cells after meiosis. The increasing accumulation of lipofuscin in these cells demarcates aging. Since lipofuscin is produced by protein hydrolysis, polyunsaturated fatty acid oxidation, and polymers produced by fatty acid oxidation and glycosylation, lipofuscin can be specifically recognized in tissue sections stained by periodic acid-schiff stain and Sudan black B. In the embodiments of the present invention, “D-galactose model” was used in gerbils as an animal model to investigate the effect of Saussurea involucrate extract for anti-fatigue and anti-aging.

### D-Galactose Aging Animal Treatment

D-galactose aging animal model was performed by administering excessive D-galactose to animals to generate reactive oxygen species and to break the balance of reactive oxygen generation and elimination, so as to induce peroxidative effect. In experimental group, D-Gal (300 mg/kg, 0.1 ml/10 g.bw) was subcutaneously injected (s.c.) daily into mice for 6 weeks. In control group, Saussurea involucrate extract (30 mg/kg body weight) was administrated by syringe daily for 6 weeks, 3 hours before subcutaneous injection of D-Gal. All control animals were subcutaneously injected with saline. The animals were finally sacrified, and the plasma of each group was collected for MDA content, antioxidative enzyme activity analysis.

Saussurea involucrate Extract

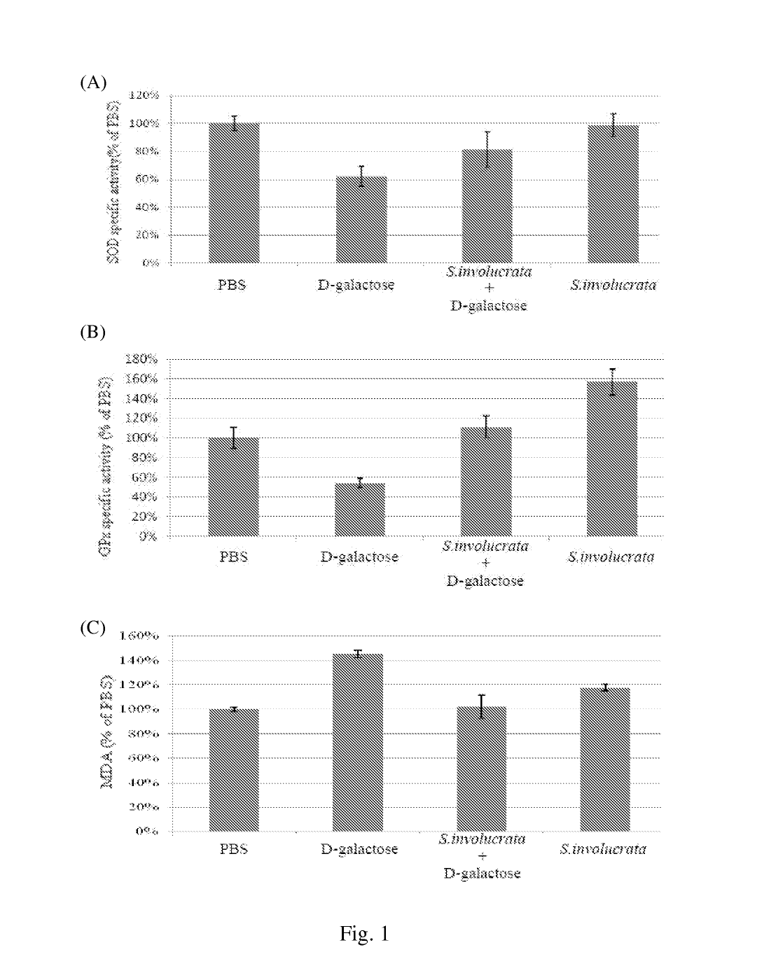

FIGS. 1(A)˜(C) show that the effect of Saussurea involucrate extract (30 mg/kg for 42 consecutive days) on neuronal damage analysis in D-galactose-treated (aged) mice, and serum superoxide dismutase (SOD), glutathione peroxidase (GPx) and malomdialdehyde (MDA) expression levels. Four groups represent PBS, D-galactose, Saussurea involucrate extract+D-galactose and Saussurea involucrate extract treated, respectively (*p<0.05 versus the D-galactose group). According to the results, long-term D-galactose treatment increases lipid peroxidation and MDA level in serum, and also decreases activities of SOD and GSH-Px, compared to those in the control group. However, administration of Saussurea involucrate extract (30 mg/kg/d) significantly inhibits MDA level and increases the activities of SOD and GSH-Px in serum. In this embodiment, ethyl acetate fraction of Saussurea involucrate extract is used as a preferred embodiment, and not limited to this fraction, pentene and n-butanol fractions also possess similar effect in different level.

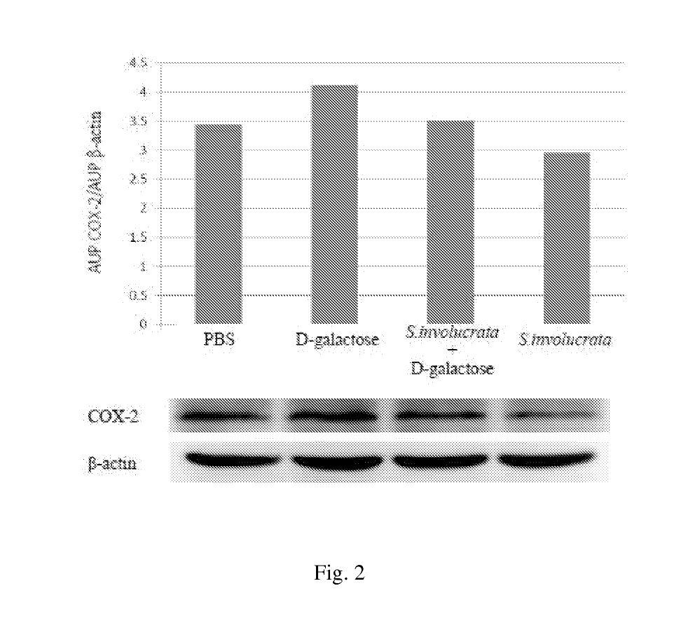

FIG. 2 shows the effect of Saussurea involucrate extract (30 mg/kg) on the D-gal-treated mice, and COX-2 expression level by western blotting. Previous studies reported that NF-kB and AP-1 can regulate COX-2 expression. Therefore, NF-kB and AP-1protein levels are measured and shown in this embodiment. Saussurea involucrate is shown to remarkably inhibit COX-2 expression (control: β-actin). These results indicate that Saussurea involucrate decreases the expression of COX-2 via downregulation of NF-kappa B.

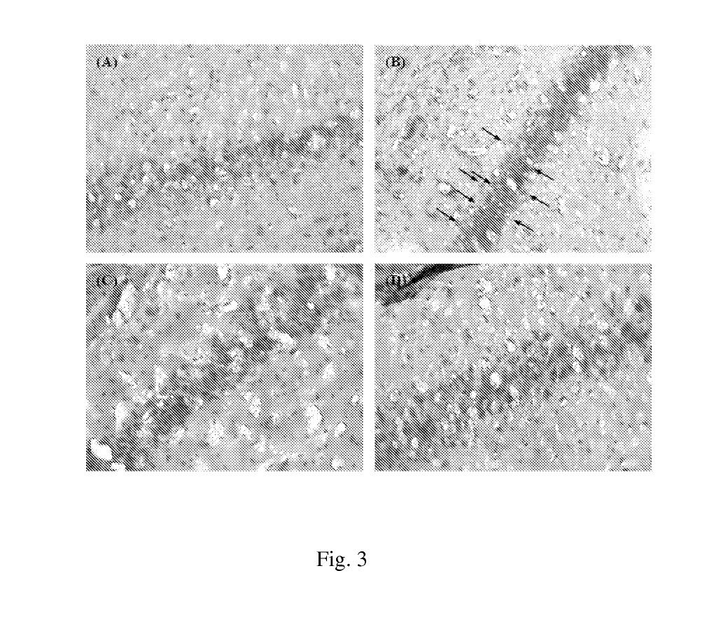

FIGS. 3(A)˜(D) show the effect of Saussurea involucrate (30 mg/kg) for apoptosis in the hippocampus of D-gal-treated mice, and cleaved caspase-3 expression level by Immunohistochemistry (IHC) staining. FIGS. 3(A)˜(D) represent PBS, D-galactose, Saussurea involucrate+D-galactose and Saussurea involucrate-treated, respectively. Immunohistochemistry (IHC) staining is used to quantify cleaved caspase-3 expression level within hippocampus. As shown in figures, treatment of cells with D-galactose induces apoptosis, which is inhibited by pretreatment with Saussurea involucrate. Treatment with Saussurea involucrate (30 mg/kg) significantly (p<0.05) inhibits the D-galactose-induced increase in Caspase-3. According to these results, it is concluded that Saussurea involucrate extract can inhibit apoptosis induced by D-galactose.

### Rutin

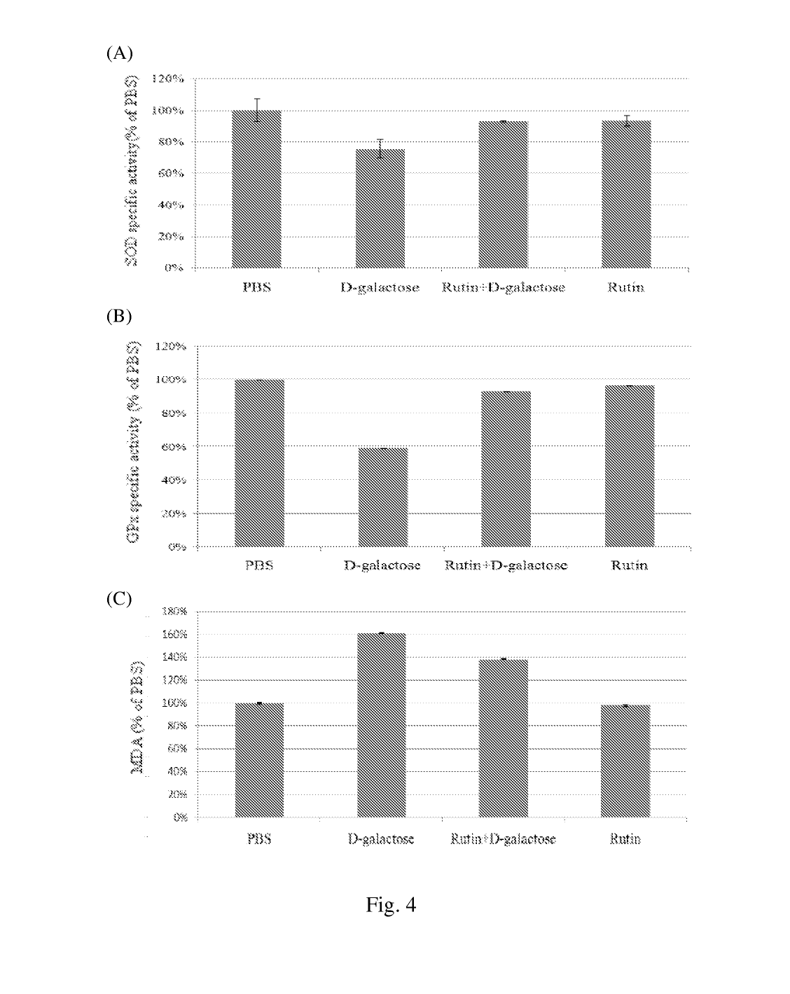

FIGS. 4(A)˜(C) show that the effect of Rutin (30 mg/kg for 42 consecutive days) on neuronal damage analysis in D-galactose-treated (aged) mice, and serum superoxide dismutase (SOD), glutathione peroxidase (GPx) and malomdialdehyde (MDA) expression levels. Four groups represent PBS, D-galactose, Rutin+D-galactose and Rutin treated, respectively (*p<0.05 versus the D-galactose group). Rutin is a compound extracted from ethyl acetate fraction and used herein as a further example. According to the results, long-term D-galactose treatment increases lipid peroxidation and MDA level in serum, and also decreases activities of SOD and GSH-Px, compared to those in the control group. However, administration of Rutin (30 mg/kg/d) significantly inhibits MDA level and increases the activities of SOD and GSH-Px in serum.

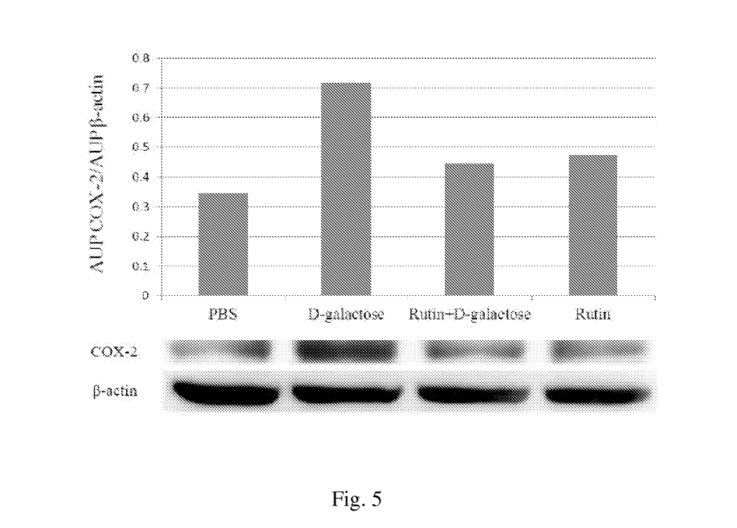

FIG. 5 shows the effect of Rutin (30 mg/kg) on the D-gal-treated mice, and NF-kappaB and COX-2 expression levels by western blotting. Previous studies reported that NF-kB and AP-1 can regulate COX-2 expression. Therefore, NF-kB and AP-1protein levels are measured and shown in this embodiment. Rutitn is shown to remarkably inhibit COX-2 expression (control: (3-actin). These results indicate that Rutin decreases the expression of COX-2 via downregulation of NF-kappa B.

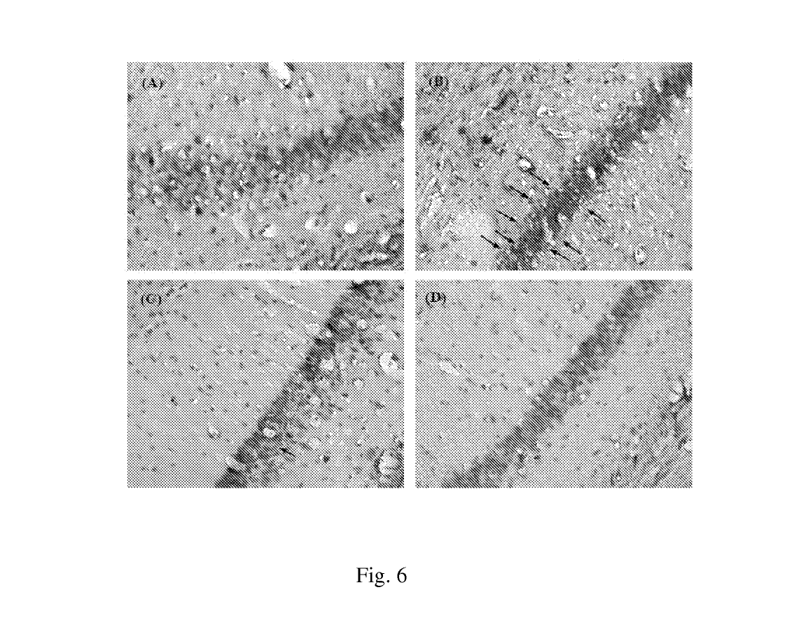

FIGS. 6(A)˜(D) show the effect of Rutin (30 mg/kg) for apoptosis in the hippocampus of D-gal-treated mice, and cleaved caspase-3 expression level by Immunohistochemistry (IHC) staining. FIGS. 6(A)˜(D) represent PBS, D-galactose, Rutin+D-galactose and Rutin-treated, respectively. As shown in figures, treatment of cells with D-galactose induces apoptosis, which is inhibited by pretreatment with Rutin. Treatment with Rutin (30 mg/kg) significantly (p<0.05) inhibits the D-galactose-induced increase in Caspase-3. According to theses results, it is concluded that Saussurea involucrate extract can inhibit apoptosis induced by D-galactose.

### Embodiment 2

### Weight-Loaded Swimming Test

In order to determine ethyl acetate extract of Saussurea involucrata has anti-fatigue activity, weight-loaded swimming test is performed. After swimming test, glycogen, urea nitrogen, and lactic acid in serum are measured for fatigue generation in prolonged periods, or fatigue elimination after exercise. Four groups including administration at the doses of 0, 50, 150 and 250 mg/kg of the ethyl acetate extract of Saussurea involucrate respectively. Initial body weight, final body weight and swimming time are measured as shown in table 1. On the other hand, post-exercise fatigue elimination is also evaluated in four groups in similar conditions as above groups. Serum urea nitrogen and lactic acid, and post-exercise lactic acid treated with or without Saussurea involucrate extract are measured to calculate the ratio of lactic acid elimination as shown in table 2. According to the results, administration of ethyl acetate extract at doses of 150 and 250 mg/kg can significantly increase swimming time of mice and ratio of lactic acid elimination. It is concluded that demonstrates that Saussurea involucrate extract has anti-fatigue activity.

TABLE 1

| | | Body | Body | |
|---|---|---|---|---|
| | Dose | weight | weight | Swimming |
| Group | (mg/kg) | (initial) | (final) | time |
| Control | | 22.6 ± 1.33 | 27.2 ± 1.92 | 16.35 ± 4.54 |
| Group 2 | 50 | 22.7 ± 1.56 | 25.2 ± 2.04 | 19.26 ± 4.41 |
| Group 3 | 150 | 23.8 ± 1.32 | 25.7 ± 0.82 | 21.63 ± 4.54 |
| Group 4 | 250 | 23.5 ± 1.43 | 24.8 ± 1.03 | 29.46 ± 4.96 |

TABLE 2

| | | Serum urea | | |
|---|---|---|---|---|
| | Dose | nitrogen | Serum lactic acid | Ratio of lactic |
| Group | (mg/kg) | (mmol/L) | (mmol/Lmin) | acid elimination |
| Control | | 17.77 ± 1.17 | 68.49 ± 18.66 | 0.0348 |
| Group 2 | 50 | 15.10 ± 2.57 | 51.67 ± 12.55 | 0.0625 |
| Group 3 | 150 | 13.10 ± 2.16 | 50.23 ± 17.17 | 0.1565 |
| Group 4 | 250 | 13.07 ± 1.27 | 44.13 ± 6.46 | 0.1402 |

Above all, the present invention discloses a Saussurea involucrate extract for anti-fatigue and anti-aging, produced by following steps: (A) extracting Saussurea involucrate with a first solvent and evaporating the first solvent to form a solid phase; (B) adding a predetermined amount of water into the solid phase to form a suspension; (C) adding a second solvent into the suspension to form an aqueous layer and an organic layer; and (D) evaporating the organic layer to obtain the Saussurea involucrate extract. Preferably, the first solvent in step (A) is methanol, the second solvent in step (B) is pentane, ethyl acetate or butanol, the Saussurea involucrate extract at least comprises a flavonoid compound which is preferably Rutin.

The present invention also discloses a use of Saussurea involucrate extract in the manufacture of a medicament for anti-fatigue and anti-aging, comprising the steps of administering a therapeutically effective amount of the Saussurea involucrate extract to a subject for anti-fatigue and anti-aging in need thereof. The Saussurea involucrate extract can inhibit malomdialdehyde expression level, increase lactic acid elimination, increase activity of superoxide dismutase and glutathione peroxidase, and inhibit COX-2 and caspase-3 expression levels.

The present invention also discloses a pharmaceutical composition for anti-fatigue and anti-aging, comprising the Saussurea involucrate extract and a pharmaceutically acceptable diluent, excipient or carrier. The composition is in the form selected from the group consisting of an intravenous injection agent, nasal agent, inhalation agent, sublingual tablet, suppository, topical injection agent, transdermal medication, oral medicine, ophthalmic agent, muscular injection agent, subcutaneous injection agent, intracutaneous injection, slow release formulation and control release formulation.

In conclusion, the present invention discloses that Saussurea involucrate extract can inhibit MDA expression level and activate glutathione peroxidase, at a lower concentration than prior art, to protect neuron cells. This means that Saussurea involucrate extract have not only therapeutical effect on central nervous system diseases, but also on apoptosis associated diseases in peripheral tissue (e.g. transplant rejection, heart failure, cardiomyopathy, ischemia-reperfusion injury of myocardium, liver, or kidney, glossdynia, myocardial infarction, radiation damage, peripheral arterial disease, peripheral circulatory insufficiency, pressure sore, corneal injury, autoimmune disease, or immunodeficiency disorders), or can be used for protection of transplanted organs and tissues. For above peripheral tissue diseases, sufficient effect can be obtained by fewer dose of the Saussurea involucrate extract than that used for nerve diseases in the prior art. In the present invention, the pharmaceutical composition is in the form selected from the group consisting of an intravenous injection agent, nasal agent, inhalation agent, sublingual tablet, suppository, topical injection agent, transdermal medication, oral medicine, ophthalmic agent, muscular injection agent, subcutaneous injection agent, and intracutaneous injection. Further, the composition can be used by mixing slow release formulation and control release formulation. In the future, Saussurea involucrate extract of the present invention can be used in neuroprotection, particularly in treatment or prevention of cerebral diseases, nerve diseases, neurodegenerative diseases, chronic neurodegenerative diseases, aging, and fatigue, caused by oxidative injury.

All of the references cited herein are incorporated by reference in their entirety.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A Saussurea involucrate extract for anti-fatigue and anti-aging, produced by following steps:
(A) extracting Saussurea involucrate with a first solvent and evaporating the first solvent to form a solid phase;
(B) adding a predetermined amount of water into the solid phase to form a suspension;
(C) adding a second solvent into the suspension to form an aqueous layer and an organic layer; and
(D) evaporating the organic layer to obtain the Saussurea involucrate extract.

2. The Saussurea involucrate extract as claimed in claim 1, wherein the first solvent in step (A) is methanol.

3. The Saussurea involucrate extract as claimed in claim 1, wherein the second solvent in step (B) is pentane, ethyl acetate or butanol.

4. The Saussurea involucrate extract as claimed in claim 1, at least comprising a flavonoid compound.

5. The Saussurea involucrate extract as claimed in claim 4, wherein the flavonoid compound is Rutin.

6. A use of Saussurea involucrate extract in the manufacture of a medicament for anti-fatigue and anti-aging, comprising the steps of administering a therapeutically effective amount of the Saussurea involucrate extract as claimed in claim 1 to a subject for anti-fatigue and anti-aging in need thereof.

7. The use as claimed in claim 6, wherein the Saussurea involucrate extract inhibits malomdialdehyde (MDA) expression of the subject.

8. The use as claimed in claim 6, wherein the Saussurea involucrate extract promotes the elimination of lactic acid of the subject.

9. The use as claimed in claim 6, wherein the Saussurea involucrate extract increases the superoxide dismutase (SOD) activity of the subject.

10. The use as claimed in claim 6, wherein the Saussurea involucrate extract increases the glutathione peroxidase (GPx) activity of the subject.

11. The use as claimed in claim 6, wherein the Saussurea involucrate extract decreases cyclooxygenase 2 (COX-2) expression of the subject.

12. The use as claimed in claim 6, wherein the Saussurea involucrate extract decreases caspase-3 expression of the subject.

13. A pharmaceutical composition for anti-fatigue and anti-aging, comprising the Saussurea involucrate extract as claimed in claim 1 and a pharmaceutically acceptable diluent, excipient or carrier.

14. The pharmaceutical composition as claimed in claim 13, wherein the composition is in the form selected from the group consisting of an intravenous injection agent, nasal agent, inhalation agent, sublingual tablet, suppository, topical injection agent, transdermal medication, oral medicine, ophthalmic agent, muscular injection agent, subcutaneous injection agent, intracutaneous injection, slow release formulation and control release formulation.
